(19) United States
(12) Patent Application Publication
(10) Pub. No.: US 2026/0284298 A1
(43) Pub. Date: Sep. 24, 2026

(54) MIXING SYRINGE ASSEMBLIES AND METHODS

(71) Applicant: CLEARSTREAM TECHNOLOGIES LIMITED, Enniscorthy, County Wexford (IE)

(72) Inventor: Victor Bell, Dublin (IE)

(73) Assignee: CLEARSTREAM TECHNOLOGIES LIMITED, Enniscorthy, County Wexford (IE)

(21) Appl. No.: 19/473,676

(22) PCT Filed: Apr. 26, 2024
(86) PCT No.: PCT/US2024/026523

§ 371 Date: Oct. 8, 2025

Related U.S. Application Data
(60) Provisional application No. 63/498,670, filed on Apr. 27, 2023.

(51) Int. Cl. A61M 5/14; A61N 5/10

(52) U.S. Cl. CPC A61M 5/1409; A61N 5/1007; A61N 2005/1021

(57) ABSTRACT

A mixing syringe assembly includes a first barrel body portion, a second barrel body portion, and a sealing device. The first barrel body portion defines a first constituent material section. The second barrel body portion defines a second constituent material section. The first barrel body portion is slidably disposed within the second barrel body portion. The sealing device is coupled to a distal end of the first barrel body portion and selectively provides a fluid path between the first barrel body portion and the second barrel body portion. The sealing device includes a first portion defining a fluid passageway which is selectively actuatable between a radially closed configuration and a radially open configuration and a second portion being axially translatable between an axially closed configuration and an axially open configuration.

### CROSS-REFERENCE TO RELATED APPLICATIONS

The present application claims priority to U.S. Patent Application No. 63/498,670, filed Apr. 27, 2024, entitled “MIXING SYRINGE ASSEMBLIES AND METHODS OF MIXING CONSTITUENT MATERIALS,” the entirety of which is hereby incorporated by reference herein.

### BACKGROUND

### Field

The present disclosure generally relates to mixing syringe assemblies and methods and, more particularly, mixing syringe assemblies for mixing and delivering material to form radiation spacers and associated methods.

### Technical Background

Prostate cancer is the most common non-skin cancer diagnosed in men. Radiation therapy is an excellent treatment option for prostate cancer. However, radiation exposure can cause unintended side effects in adjacent non-targeted tissue. A radiation spacer, such as a radio protective spacer) can be implanted to avoid collateral radiation and minimize injury to nearby tissues by providing a space between the target tissue and non-targeted tissues at risk.

Conventional radiation spacers may include gelling materials, which are delivered as a liquid and allowed to cure. However, constituent components of the gel may begin to gel on contact with one another, which may make delivery difficult. Moreover, constituent components may need to remain separate until time of use, otherwise they may degrade over time due to limited stability.

### SUMMARY

Embodiments of the present disclosure are directed to various mixing syringe assemblies, which may maintain separation of constituent materials until time for use of the mixed constituent materials.

In one embodiment a mixing syringe assembly includes a first barrel body portion, a second barrel body portion, and a sealing device. The first barrel body portion defines a first constituent material section. The second barrel body portion defines a second constituent material section. The first barrel body portion is slidably disposed within the second barrel body portion. The sealing device is coupled to a distal end of the first barrel body portion and selectively provides a fluid path between the first barrel body portion and the second barrel body portion. The sealing device includes a first portion defining a fluid passageway which is selectively actuatable between a radially closed configuration and a radially open configuration and a second portion being axially translatable between an axially closed configuration and an axially open configuration.

In another embodiment, a mixing syringe assembly includes a first barrel body portion, a second barrel body portion, a sealing device, a plunger, and a lock. The first barrel body portion defines a first constituent material section. The second barrel body portion defines a second constituent material section. The first barrel body portion is slidably disposed within the second barrel body portion. The sealing device is coupled to a distal end of the first barrel body portion and selectively provides a fluid path between the first barrel body portion and the second barrel body portion. The sealing device includes a first portion defining a fluid passageway which is selectively actuatable between a radially closed configuration and a radially open configuration and a second portion being axially translatable between an axially closed configuration and an axially open configuration. The plunger is axially moveably within the first barrel body portion. The lock is configured to brace the plunger in a locked position.

In yet another embodiment, a method of assembling a mixing syringe assembly includes coupling a sealing device to a distal end of a first barrel body portion, wherein the sealing device comprises a first portion defining a fluid passageway which is selectively actuatable between a radially closed configuration and a radially open configuration and a second portion being axially translatable between an axially closed configuration and an axially open configuration, and inserting the sealing device and the first barrel body portion into a second barrel body portion, such that the sealing device and the first barrel body portion are slidably disposed in the second barrel body portion.

Additional features and advantages of the aspects described herein will be set forth in the detailed description, which follows, and in part will be readily apparent to those skilled in the art from that description or recognized by practicing the aspects described herein, including the detailed description, which follows, the claims, as well as the appended drawings.

It is to be understood that both the foregoing general description and the following detailed description describe various aspects and are intended to provide an overview or framework for understanding the nature and character of the claimed subject matter. The accompanying drawings are included to provide a further understanding of the various aspects, and are incorporated into and constitute a part of this specification. The drawings illustrate the various aspects described herein, and together with the description serve to explain the principles and operations of the claimed subject matter.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, wherein like structure is indicated with like reference numerals and in which:

FIG. 1A schematically depicts an embodiment of a mixing syringe assembly for mixing constituent materials that includes a first barrel body portion, a second barrel body portion and a sealing device, according to one or more embodiments shown and described herein;

FIG. 1B schematically depicts movement of a plunger in the first barrel body portion of FIG. 1A, according to one or more embodiments shown and described herein;

FIG. 1C schematically depicts movement of the first barrel body portion and the plunger of FIG. 1B within the second barrel body portion, according to one or more embodiments shown and described herein;

FIG. 2A illustrates a more detailed view of the sealing device of FIG. 1A, according to one or more embodiments shown and described herein;

FIG. 2B depicts the sealing device on the end of the first barrel body portion without he second barrel body portion of FIG. 2A, according to one or more embodiments shown and described herein;

FIG. 2C depicts a cross-sectional view of a portion of the sealing device in isolation, according to one or more embodiments shown and described herein;

FIG. 3A schematically depicts a cross-sectional view of the sealing device of FIG. 2C in a closed configuration, according to one or more embodiments shown and described herein;

FIG. 3B schematically depicts actuation of the sealing device of FIG. 3A to an open configuration, according to one or more embodiments shown and described herein;

FIG. 4A depicts a dual barrel mixing syringe assembly, according to one or more embodiments shown and described herein;

FIG. 4B depicts a cross-sectional view of the dual barrel mixing syringe assembly of FIG. 4A, according to one or more embodiments shown and described herein;

FIG. 4C depicts a plunger of the dual barrel mixing syringe assembly of FIG. 4B, actuated to a first position, according to one or more embodiments shown and described herein;

FIG. 4D depicts the plunger and a first barrel body portion of the dual barrel mixing syringe assembly of FIG. 4C actuated to a second position, according to one or more embodiments shown and described herein; and

FIG. 5 depicts a flow chart illustrating a method of assembling a mixing syringe assembly, according to one or more embodiments shown and described herein.

### DETAILED DESCRIPTION

The present disclosure is generally directed to mixing syringe assemblies for mixing at least two constituent materials. Moreover, particularly the present application is directed to mixing syringe assemblies configured to delivering a radiation spacer. For example, radiation spacers may be formed of one or more hydrogel materials, which may be delivered to a desired location, such as within a balloon or on its own and cured in place to block or substantially block radiation, which may be unintentionally directed toward healthy tissue instead of targeted/diseased tissue. During mixing of the hydrogel for forming a radiation spacer, multiple materials may need to be combined just prior to and/or at delivery of the radiation spacer into a target location within the body. To maintain quality of material, powder components may need to be kept dry or unconstituted prior to application. However, during use, powder components may need to be hydrated or mixed with a diluent. It may be difficult to determine proper mixing volumes, maintain sterility, and prevent unwanted early mixing using traditional methods. Embodiments of the present disclosure may provide constituent materials in prepackaged, pre-measured, ready-to use syringe assemblies, which, as will be described in greater detail herein, provide improved mixing, maintains sterility, and/or improves delivery.

Particular embodiments of a mixing syringe assembly are directed to a nested barrel body design including a first barrel body portion, a second barrel body portion, and a sealing device. The first barrel body portion and the second barrel body portion may separate constituent material sections which are maintained in isolation from one another via the sealing device until the sealing device is actuated from a closed configuration to an open configuration. That is, the sealing device prevents unwanted moisture transfer between the first barrel body portion and the second barrel body portion. For example, of the present disclosure includes two sealing mechanisms, including a first portion which seals in a radial direct and a second portion which seals in an axial direction, thereby providing improved sealing between the constituent material sections of the first barrel body portion and the second barrel body portion. These and additional benefits and embodiments will be described in greater detail below.

Directional terms as used herein—for example up, down, right, left, front, back, top, bottom—are made only with reference to the figures as drawn and are not intended to imply absolute orientation unless otherwise specified.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any device or assembly claim does not actually recite an order or orientation to individual components, or it is not otherwise specifically stated in the claims or description that the steps are to be limited to a specific order, or that a specific order or orientation to components of an device or assembly is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation; and the number or type of embodiments described in the specification.

As used herein, the singular forms “a,” “an” and “the” include plural referents unless the context clearly dictates otherwise. Thus, for example, reference to “a” component includes aspects having two or more such components, unless the context clearly indicates otherwise.

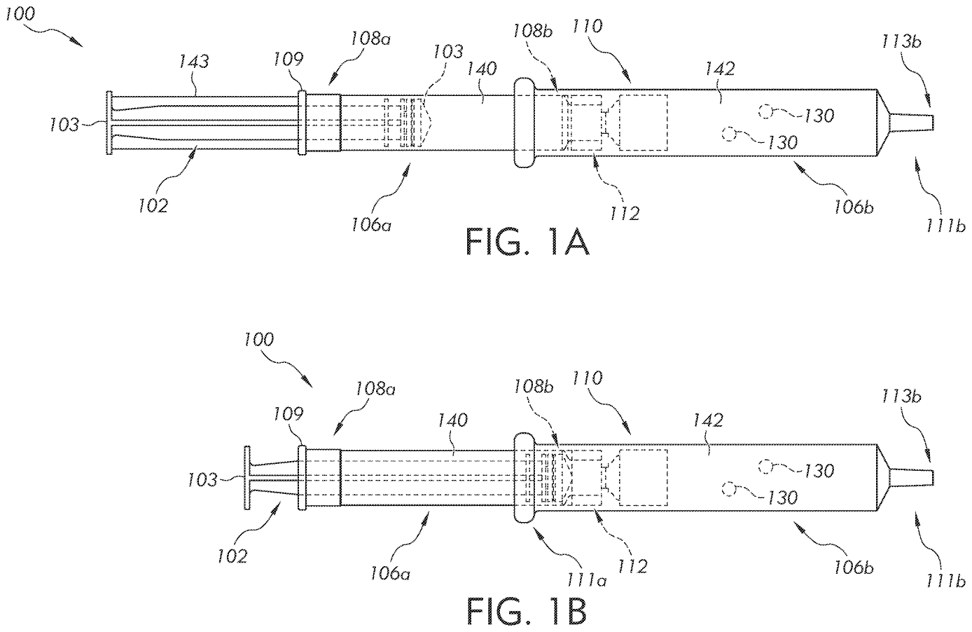

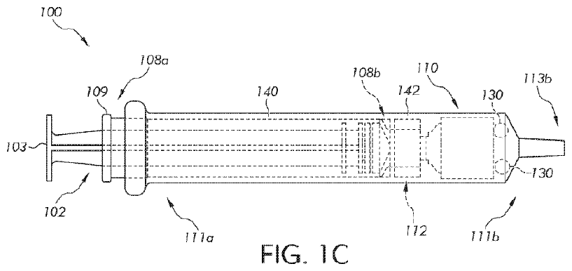

Turning now to the drawings, FIGS. 1A-1C depict an illustrative mixing syringe assembly 100. The mixing syringe assembly 100 may be used for combining two or more constituent materials as will be described in greater detail herein. In particular, the mixing syringe assembly 100 may be used to mix or combine two or more constituent components of a hydrogel for use as a radiation spacer. The mixing syringe assembly 100 generally includes a first barrel body portion 106a, a second barrel body portion 106b, and a sealing device 110. In some embodiments, the mixing syringe assembly 100 further includes a plunger 102, a plunger lock 143, and/or one or more mixing masses 130. It is noted that a mixing syringe assembly 100 may include a greater or fewer number of components without departing from the scope of the present disclosure.

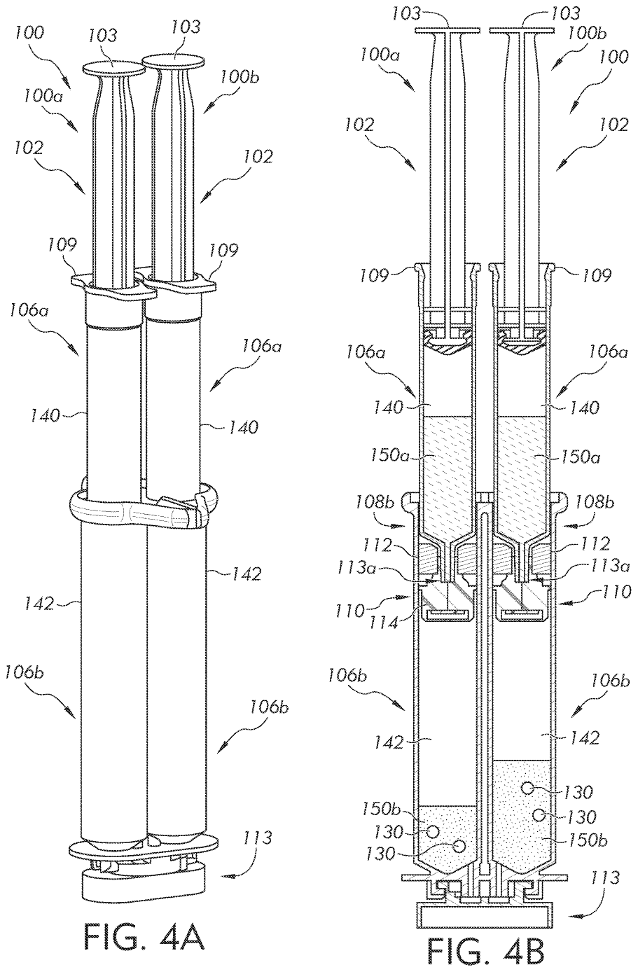

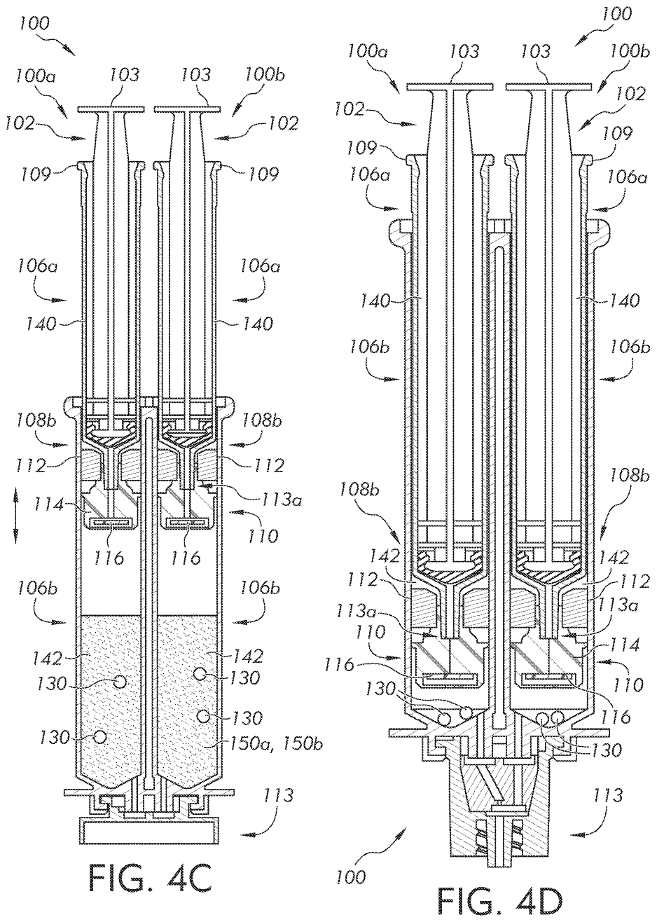

The first barrel body portion 106a may be a hollow syringe barrel defining a first constituent material section 140 or volume therein for holding a first constituent material. The first barrel body portion 106a may have a proximal end 108a and a distal end 108b. The proximal end 108a may be sized and shaped to receive the plunger 102 into the first constituent material section 140. The first barrel body portion 106a may include a first fluid delivery opening 113a (depicted in FIG. 4A-4D) located at the distal end 108b of the of the first barrel body portion 106a. At the proximal end 108a may be a first flange 109. That is, the first flange 109 may extend outward, such as radially outward, at the proximal end 108a.

The second barrel body portion 106b may also be a hollow syringe barrel defining a second constituent material section 142 or volume therein for holding a second constituent material. The second barrel body portion 106b may have a proximal end 111a and a distal end 111b. Similar to the first barrel body portion 106a, the second barrel body portion 106b may include a second fluid delivery opening 113b at the distal end 111b, which may be coupled to a material delivery cannula (not shown, which may include a manifold, delivery needle or the like). In embodiments, the second fluid delivery opening 113b may be axially positioned with a centerline of the second barrel body portion 106b and/or plunger 102. However, in other embodiments, the second fluid delivery opening 113b may be offset from a centerline of the second barrel body portion 106b and/or plunger 102. Such offset may be useful in preventing the one or more mixing masses 130 from blocking the fluid delivery opening 113b.

As depicted, the first barrel body portion 106a is slidably disposed within the second barrel body portion 106b such that the first barrel body portion 106a may telescope within the second barrel body portion 106b. That is, the first barrel body portion 106a may have a smaller diameter than the second barrel body portion 106b. For example, the first barrel body portion 106a may be a 5 ml barrel and the second barrel body portion 106b may be a 10 ml barrel body, though other sizes are contemplated and possible.

The plunger 102 may be slidably disposed in the first constituent material section 140 of the first barrel body portion 106a and may form a fluidic seal 104 with the first barrel body portion 106a via a seal 104 at a distal end 108b of the plunger 102. For example, the seal 104 may be a rubber or similar material conformable material for forming a fluid-tight seal with the first barrel body portion 106a. At a proximal end 108a of the plunger 102 may be a pusher flange 103 for a user to engage for advancing the plunger 102 along the first barrel body portion 106a.

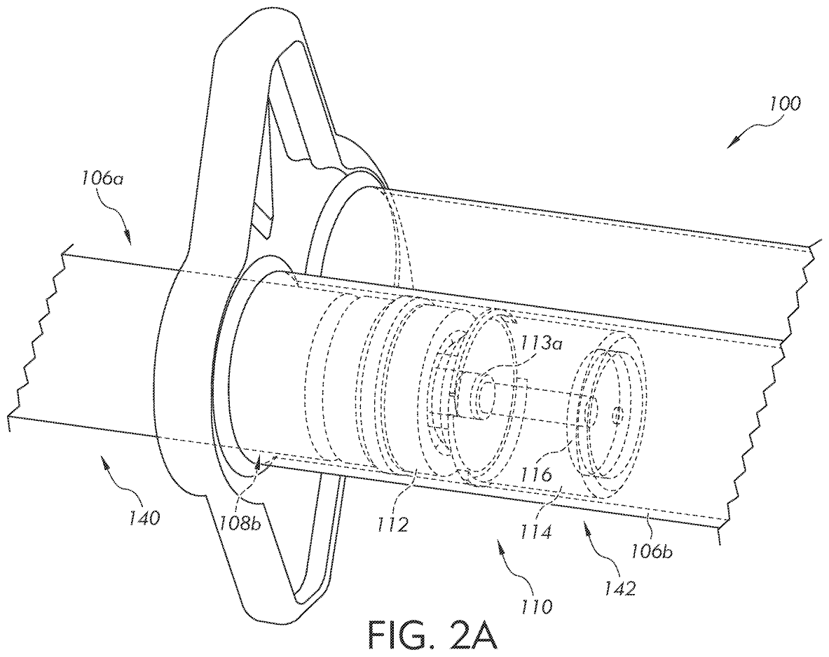

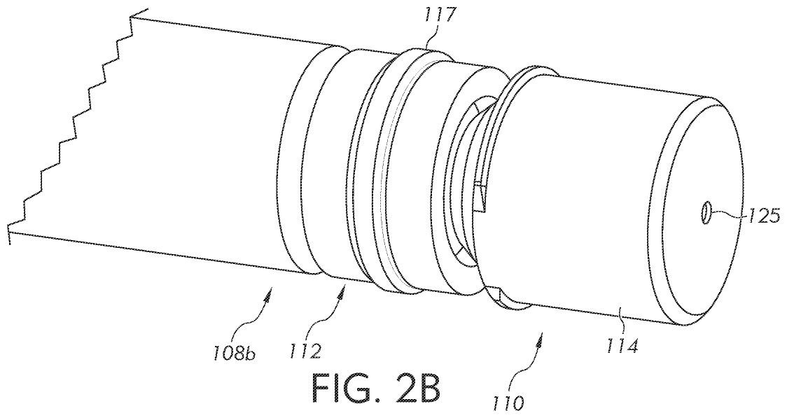

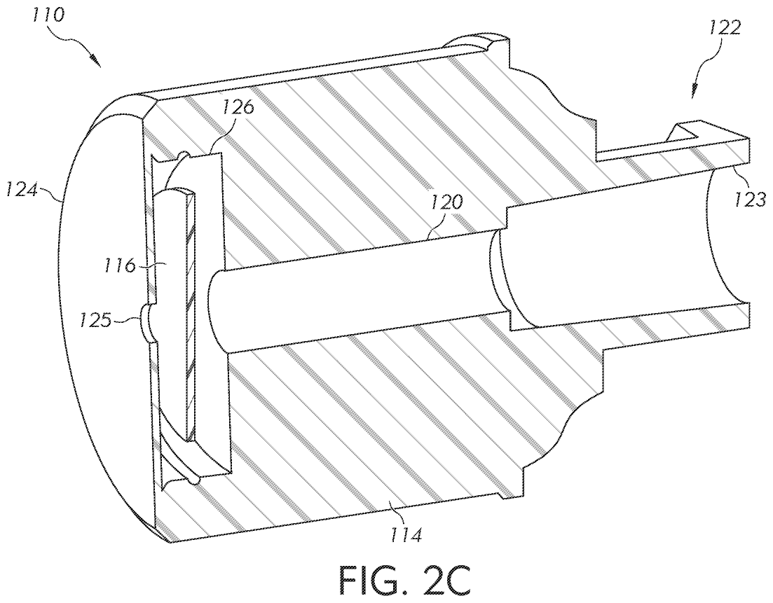

In the present embodiment, the sealing device 110 may be coupled to the distal end 108b of the first barrel body portion 106a such as around the first fluid delivery opening 113a. For example, the sealing device 110 may threadingly engage or pressfit on to the distal end 108b of the first barrel body portion 106a around the first fluid delivery opening 113a. In embodiments, any number of sealing materials, O-rings, or the like may be used to prevent leakage around the coupling of the sealing device 110 to the first barrel body portion 106a. Referring briefly to FIG. 2A-2C, the sealing device 110 includes a first portion 114 defining a fluid passageway 120, which is selectively actuatable between a radially closed configuration and a radially open configuration and a second portion 116, which is axially translatable between an axially closed configuration and an axially open configuration. In some embodiments, the sealing device 110 may further include a housing 112.

In embodiments, the housing 112 may be a collar which couples to the first portion 114. In some embodiments, it is contemplated that the housing 112 may couple the first portion 114 to the distal end 108b of the first barrel body portion 106a. For example, the first portion 114 of the sealing device 110 may be positioned within the housing 112 and coupled thereto via an interlocking engagement, pressfit, threaded connection, or the like. Similarly, the housing 112 may couple the first barrel body portion 106a. For example, the housing 112 may threadingly engage or pressfit on to the distal end 108b of the first barrel body portion 106a around the first fluid delivery opening 113a. In embodiments, any number of sealing materials, O-rings, or the like may be used to prevent leakage around the coupling of the housing 112 to the first portion 114 of the sealing device 110 and to the distal end 108b of the first barrel body portion 106a. The coupling, accordingly may provide a flow path from the first fluid delivery opening 113a to the fluid passageway 120 of the first portion 114 of the sealing device 110. In some embodiments, the first portion of the sealing device 110 may receive the distal end 108b of the first barrel body portion 106a and be couple thereto, such as via pressfit, threaded engagement, or the like. In embodiments, sealing device 110 may include an O-ring 117 mounted to the housing 112 and in sliding contact with the second barrel body portion 106b. The O-ring 117 may prevent fluid leakage in a proximal direction around the sealing device 110.

FIG. 2C provides a longitudinal cross-section of a portion of the sealing device 110. In particular, a valve portion of the sealing device 110. The valve portion includes the first portion 114 and the second portion 116. The first portion 114 may be formed of a conformable material or radially compressible material, such as silicone or the like, and may be molded or otherwise formed to have a fluid passageway 120. For example, the fluid passageway 120 may be cut through the body of the first portion 114. In the depicted embodiment, the fluid passageway 120 is illustrated in a radially open configuration. For example, fluid pressure, such as caused via actuation of the plunger 102, may force the fluid passageway 120 to the radially open configuration.

Still referring to FIG. 2C the first portion 114 have a mounting portion 122 for mounting to or adjacent to the first fluid delivery opening 113a such as to receive fluid from the first fluid delivery open. For example, in embodiments, proximal to the fluid passageway 120 may be a nozzle-receiving recess 123, for receiving the distal end 108b of the first barrel body portion 106a (such as a nozzle of the distal end 108b). In embodiments, the mounting portion 122 may be sized to be received within the housing 112.

In embodiments, the first portion 114 includes a delivery portion 124 distal to the mounting portion 122. The delivery portion 124 may have an increased diameter relative to the mounting portion 122. The increased diameter may provide increased stability within the first barrel body portion 106a. Formed at the end of the delivery portion 124 and in fluidic communication with the fluid passageway 120 may be a delivery outlet opening 125. As depicted, the second portion 116 may be positioned within the first portion 114. For example, in fluidic communication with the fluid passageway 120 may be a cavity 126 sized to hold the second portion 116 and allow axial movement thereof.

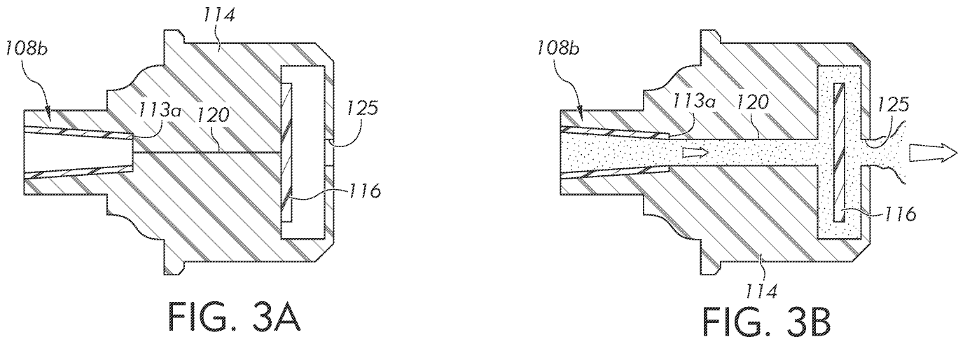

In particular, the second portion 116 may be axially translatable between an axially closed configuration and an axially open configuration. That is, the second portion 116 may be a substrate, plate, disc, or the like formed of a non-permeable membrane such as, but not limited to polyisoprene. The cavity 126 may be sized to allow the second portion 116 to axially translate therein under pressure. For example, and with reference to FIGS. 3A and 3B the valve portion is shown in a closed configuration and an open configuration, respectively. As illustrated in FIG. 3A, in the closed configuration, the fluid passageway 120 is radially closed to the radially closed configuration. In embodiments, the valve material (e.g., silicone or similar material) may tightly compacted or naturally biased to close off the fluid passageway 120 to the radially closed configuration. For example, the fluid passageway 120 may be provided via a linear cut as opposed to a punch where material is removed. In the radially closed configuration, fluid is substantially blocked from flowing through the fluid passageway 120. Accordingly, in the open configuration, fluid may exert pressure (e.g., via actuation by the plunger 102 moving distally within the first barrel body portion 106a) on the fluid passageway 120 to radially open or actuate the fluid passageway 120 to the radially open configuration thereby allowing fluid through the fluid passageway 120, as depicted in FIG. 3B.

Referring again to FIG. 3A, in the closed configuration, the second portion 116 may be positioned in the axially closed configuration. That is, the second portion 116 may be pressed against the fluid passageway 120 (when in the radially closed configuration). In the axially closed configuration, the second portion 116 acts as a second barrier to prevent fluid leakage. The second portion 116 may couple or attach to the first portion 114 similar to a suction cup engagement. In some embodiments, the second portion 116 may be biased to the axially closed configuration such as via a spring or other biasing mechanism. In some embodiments, the second portion 116 is pulled into engagement with the first portion 114 such as by retracting the plunger 102 in the proximal direction. During delivery, the plunger 102 within the first barrel body portion 106a actuates the first portion 114 of the sealing device 110 to the radially open configuration and the second portion 116 to the axially open configuration. Stated another way, the plunger 102 may be actuated in the distal direction to push fluid within the first barrel body portion 106a through the sealing device 110 to thereby open the fluid passageway 120. Opening of the fluid passagway and/or the force of fluid impinging on the second portion 116 may cause the second portion 116 to move axially away from the fluid passageway 120 to allow fluid to flow around the second portion 116 and out of the delivery outlet opening 125 (and into the second barrel body portion 106b) positioned distal to the second portion 116. When force is not applied via the plunger 102, the first portion 114 may close to cause the fluid passageway 120 to move to the radially closed configuration. Similarly, the second portion 116 may return to the axially closed configuration. For example, the second portion 116 may be biased to the axially closed configuration, or any backflow may force the second portion 116 to the closed configuration to prevent backflow through the sealing device 110.

In some embodiments, and as noted above, the mixing syringe assembly 100 may include a plunger lock 143. With reference to FIG. 1A, the plunger lock 143 may lock a position of the plunger 102 relative to the first barrel body portion 106a, the second barrel body portion 106b, or both. In some embodiments, the lock 143 may include one or more struts which extend between the first barrel body portion 106a and the pusher flange 103. In some embodiments, the lock 143 may be a collar which partially wraps around the plunger 102 and extends between the first barrel body portion 106a and the pusher flange 103 to lock the plunger 102 in place. In some embodiments, the plunger lock 143 may be used to maintain the plunger 102 in a position causing a negative pressure on the sealing device 110 (to force a tighter compression within the first portion 114 and/or between the first portion 114 and the second portion 116) for added safety and moisture transfer prevention. During use the plunger lock 143 may be removed prior to advancing the plunger 102.

As noted above, the first barrel body portion 106a defines a first constituent material section 140. The first constituent material section 140 may hold a first constituent material. The first constituent material may be a liquid for hydrating a second constituent material, which may be a powder or particulate. The second constituent material may be held within the second constituent material section 142 of the second barrel body portion 106b. For example, the first constituent material may be saline, water, deionized water, or the like. As noted above, the second constituent material may be a powder or particulate material such as but not limited to albumin, polyethylenimine (PEI), an amine containing polyethylene glycol (PEG) or protein, an N-hydroxysuccinimide (NHS) ester component such as PEG-(SS)2, PEG-(SS)4, PEG-(SS)8, PEG-(SG)4, PEG-(SG)8, and/or the like. In some aspects, molecular weights of the PEG components may range from about 2,000 to about 100,000. The powder or particular material may be biodegradable and/or bioabsorbable. As used herein, “biodegradable” and/or “bioabsorbable” refers to a compound that can be absorbed by the surrounding or local tissue of a subject and/or degraded and absorbed by the tissue of the subject.

The powder or particulate material can be composed of various crosslinking substances of varying amounts, designed to allow the hydrogel to last a specific amount of time in situ before degrading. In aspects, the hydrogel components may be selected based on a degradation time that corresponds to the length of anticipated radiation therapy. In aspects the length of anticipated radiation therapy, and thus the targeted time for hydrogel degradation is up to 18 months 9, for example from the range of about 0 months 9 to about 18 months 9, including about 1 month, 2 months 9, 3 months 9, 4 months 9, 5 months 9, 6 months 9, 7 months 9, 8 months 9, 9, months 9, 10 months 9, 11 months 9, 12 months 9, 13 months 9, 14 months 9, 15 months 9, 16 months 9, 17 months 9, and 18 months 9. It should be understood that the time is merely a rough guide generally used to target appropriate formulation of the hydrogel.

The sealing device 110 described above is actuated to selectively provide a flow path between the first constituent material section 140 and the second constituent material section 142 to allow for the first constituent material from the first constituent material section 140 to move into the second constituent material section 142 to allow for mixing of the first constituent material with the second constituent material.

Referring again to FIGS. 1A-1C, in some embodiments, one or more mixing masses 130 (e.g., balls, weights, etc.) may positioned within the second constituent material section 142. The one or more mixing masses 130 may be any suitable material to assist in mixing materials such as stainless steel, delron, plastic polycarbonate, composite, aluminum, low carbon steel, chemical-resistant PTFE, composite, titanium, moisture resistant polyethylene, magnetic material, etc. In one or more mixing masses 130, may be any shape such as spherical, cylindrical, cubic, ellipsoid, or any other regular or irregular shape. The one or more mixing masses 130 may include any number of masses such as two or more, three or more, four or more etc. The one or more mixing masses 130 may be identical to one another or different from one another. The one or more mixing masses 130 may have various sizes such as up to 7 mm in diameter, such as up to 6 mm in diameter, such as up to 5 mm in diameter, such as up to 4 mm in diameter, such as up to 3 mm in diameter, such as 7 mm or less in diameter. In various embodiments, the one or more mixing masses 130 may have designs cut or etched into its surface, which may enhance mixing. In some embodiments, there may be no mixing masses 130.

Referring now to FIGS. 4A-4D, in various embodiments, a mixing syringe assembly 100 may include one or more mixing syringe subassemblies, such as to provide a dual barrel syringe. For example, FIGS. 4A-4D illustrate a dual-mixing syringe assembly 100 including a first mixing syringe subassembly 100a and a second mixing syringe subassembly 100b. Each subassembly 100a, 100b is substantially identical to the mixing syringe assembly 100 described with respect to FIG. 1A-1C. In particular, each subassembly 100a may include a plunger 102, a first barrel body portion 106a, a second barrel body portion 106b, and a sealing device 110. In some embodiments, the subassemblies 100a, 100b further include one or more mixing masses 130. Accordingly, the above description is applicable to the present embodiments and will not be repeated. However, it is noted that while each plunger 102 appears separately actuatable, in embodiments, the plungers 102 may be coupled to one another via a common pusher flange. In some embodiments, the second barrel body portions 106b may be rigidly coupled. For example, the second barrel body portions 106b may be integrally formed or coupled to another via a bracket, welding, adhesive or the like. Similarly, in some embodiments, first barrel body portions 106a may be coupled to one another in a similar fashion while allowing the first barrel body portions 106a to slide within respective second barrel body portion 106b.

In some embodiments, a manifold may couple the fluid delivery openings to respective delivery needles or cannulas for delivery of fluid. It is noted that in various embodiments, it may be desirable to keep mixed constituent materials from the first subassembly 100a and the second subassembly 100b separate until the point of delivery at a target delivery location, such as within a subject. Accordingly, dual needle assemblies and/or coaxial needle assemblies such as described in International Patent Application No. PCT/US2021/023171, entitled “Multi-Component Sealant Delivery Systems Incorporating Quarter Turn Connectors,” filed Mar. 19, 2021, the entirety of which is hereby incorporated by reference, may be desirable. For example, while the first mixing syringe subassembly 100a may include the first constituent material 150a and the second constituent material 150b, the second mixing syringe subassembly 100b may hold a different first constituent material 150a and/or a different second constituent material 150b. For example, a second constituent material 150b of the first mixing syringe subassembly 100a may hold Peg 8, having 20 kmw, while the second constituent material 150b of the second mixing syringe subassembly 100b may hold Peg 8 having 15 kmw. The first constituent materials 150a may also be the same or different and may include, but are not limited to water saline, deionized water, or like. In embodiments, the resulting mixtures or solutions within each of the first and second subassemblies 100a, 100b, may gel or solidify on contact with one another. Accordingly, it may be desirable to only allow contact at the moment of delivery, such as at the end of a dual-lumen cannula, such as disclosed in International Patent Application No. PCT/US2021/023171, entitled “Multi-Component Sealant Delivery Systems Incorporating Quarter Turn Connectors,” filed Mar. 19, 2021, the entirety of which is hereby incorporated by reference.

Still referring to FIGS. 4A-4D, operation is generally depicted. FIG. 4A illustrates a perspective view of the dual syringe assembly. FIG. 4B illustrates a cross-sectional view of FIG. 4A and depicts a first constituent material 150a positioned within respective first constituent material sections 140 and a second constituent material 150b positioned within respective second constituent material sections 142. Referring to FIG. 1C, in operation, the plunger 102(s) of the assembly may be advanced building fluid pressure within the first constituent material section 140 behind the sealing device 110. Once the pressure exceeds a threshold of the first portion 114 of the sealing device 110, the fluid passageway 120 is radially actuated to an open configuration (depicted in FIG. 4C) and the second portion 116 is axially actuated to move away from the first portion 114. With both the first portion 114 and the second portion 116 actuated to an open configuration, the first constituent material 150a is allowed to pass from the first constituent material section 140 through the sealing device 110 to the second constituent material section 142 within the second barrel body portion 106b (e.g., of the respective sub-assembly). Once the first constituent material 150a enters the second constituent material section 142, the mixing syringe assembly 100 may then be agitated (e.g., shaken) to incorporate that first and second constituent materials. As noted above, the one or more mixing masses 130 may assist in agitation while reducing introduction of air bubbles. After mixing, the plunger 102 and the first barrel body portion 106a together with the sealing device 110 may be advanced together to expel the combined constituent materials out of the fluid delivery opening 113b (or respective opening in dual design). It is noted that in various embodiments, the liquid constituent material may be placed in the second constituent material section 142 and the powder or particulate component may be placed initially in the first constituent material section 140.

In embodiments, including the a dual syringe, one mixing syringe subassembly 100a may include albumin and/or microparticles as the second constituent material 150b and the other mixing syringe subassembly 100a may include PEG. Other combinations are contemplated and possible. After mixing with respective liquid constituent materials the now-liquid albumin and PEG solutions may be dispensed to form a gel at the desired location.

It is noted that in the embodiment above, while the system may be provided as completely assembled. In embodiments, the different components may be provided individually and assembled prior to use.

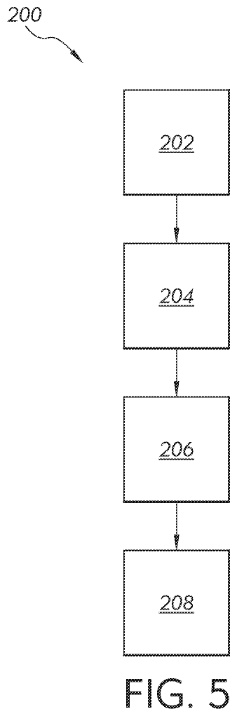

Referring now to FIG. 5 a flowchart depicting a method 200 of assembling a mixing syringe assembly 100 such as described herein is generally depicted. It is noted that the method 200 may include a greater or fewer number of steps taken in any order without departing from the scope of the present disclosure. In embodiments, the method 200 includes at block 202 coupling the sealing device 110 to a distal end 108b of the first barrel body portion 106a as described above. For example, the sealing device 110 may be coupled to the first barrel body portion 106a using any suitable technique (e.g. pressfit, threaded engagement, etc.). In some embodiments, the sealing device 110 includes a housing 112 which couples to the distal end 108b of the first barrel body portion 106a. The method 200 may further include, at block 204, inserting the sealing device 110 and the first barrel body portion 106a into the second barrel body portion 106b such that the sealing device 110 the first barrel body portion 106a are slidably disposed in the second barrel body portion 106b.

In some embodiments, the method 200 may further include, at block 206. inserting the plunger 102 into the first barrel body portion 106a. In some embodiments, the method 200 may include, at block 208, placing a first constituent material 150a, such as described above, into the first barrel body portion 106a. In embodiments, the plunger 102 may be used to first pull the first constituent material 150a into the first barrel body portion 106a prior to coupling the sealing device 110 to the first barrel body portion 106a. In some embodiments, after placing the plunger 102, a plunger lock 143 may be attached to the plunger 102 to lock a position of the plunger 102 relative to the first barrel body portion 106a.

In embodiments, the method 200 may include at block 208, placing the second constituent material 150b into the second barrel body portion 106b. In embodiments, the second constituent material 150b may be placed prior to positioning the first barrel body portion 106a within the second barrel body portion 106b.

In embodiments, the assembly mixing syringe assembly 100 may be provided in a read-to-use preassembled format with appropriately measured materials already positioned therein.

Embodiments may be further described with respect to the following numbered clauses:
1. A mixing syringe assembly comprising: a first barrel body portion defining a first constituent material section; a second barrel body portion defining a second constituent material section, the first barrel body portion slidably disposed within the second barrel body portion; and a sealing device coupled to a distal end of the first barrel body portion, the sealing device selectively providing a fluid path between the first barrel body portion and the second barrel body portion, the sealing device comprising a first portion defining a fluid passageway which is selectively actuatable between a radially closed configuration and a radially open configuration and a second portion being axially translatable between an axially closed configuration and an axially open configuration.
2. The mixing syringe assembly of clause 1, further comprising a plunger, wherein advancing the plunger within the first barrel body portion actuates the first portion of the sealing device to the radially open configuration and the second portion to the axially open configuration.
3. The mixing syringe assembly of any preceding clause, wherein the first portion of the sealing device comprises radially compressible silicone.
4. The mixing syringe assembly of any preceding clause, wherein the second portion of the sealing device comprises a polyisoprene substrate.
5. The mixing syringe assembly of any preceding clause, wherein the sealing device comprises a housing coupled to an end of the first barrel body portion, wherein the first portion is positioned within the housing.
6. The mixing syringe assembly of clause 5, wherein the sealing device further comprises an O-ring mounted to the housing and in sliding contact with the second barrel body portion.
7. A mixing syringe assembly comprising: a first barrel body portion defining a first constituent material section; a second barrel body portion defining a second constituent material section, the first barrel body portion slidably disposed within the second barrel body portion; a sealing device coupled to a distal end of the first barrel body portion, the sealing device selectively providing a fluid path between the first barrel body portion and the second barrel body portion, the sealing device comprising a first portion defining a fluid passageway which is selectively actuatable between a radially closed configuration and a radially open configuration and a second portion being axially translatable between an axially closed configuration and an axially open configuration; a plunger axially moveable within the first barrel body portion; and a lock configured to brace the plunger in a locked position.
8. The mixing syringe assembly of clause 7, wherein advancing the plunger within the first barrel body portion actuates the first portion of the sealing device to the radially open configuration and the second portion to the axially open configuration.
9. The mixing syringe assembly of clause 7 or 8, wherein the first portion of the sealing device comprises radially compressible silicone.
10. The mixing syringe assembly of any of clauses 7-9, wherein the second portion of the sealing device comprises a polyisoprene substrate.
11. The mixing syringe assembly of any of clauses 7-10, wherein the sealing device comprises a housing coupled to an end of the first barrel body portion, wherein the first portion is positioned within the housing.
12. The mixing syringe assembly of clause 11, wherein the sealing device further comprises an O-ring mounted to the housing and in sliding contact with the second barrel body portion.
13. A method of assembling a mixing syringe assembly, the method comprising: coupling a sealing device to a distal end of a first barrel body portion, wherein the sealing device comprises a first portion defining a fluid passageway which is selectively actuatable between a radially closed configuration and a radially open configuration and a second portion being axially translatable between an axially closed configuration and an axially open configuration; and inserting the sealing device and the first barrel body portion into a second barrel body portion, such that the sealing device and the first barrel body portion are slidably disposed in the second barrel body portion.
14. The method of clause 13, further comprising inserting a plunger into the first barrel body portion, wherein sliding of the plunger relative to the first barrel body portion actuates the first portion of the sealing device to the radially open configuration and the second portion to the axially open configuration.
15. The method of clause 13 or 14, further comprising placing a first constituent material into the first barrel body portion.
16. The method of clause 15, further comprising placing a second constituent material into the second barrel body portion, the second constituent material being different from the first constituent material.
17. The method of clause 16, wherein the first constituent material is a liquid and the second constituent material is a powder.
18. The method of any of clauses 13-17, further comprising: inserting a plunger into the first barrel body portion, wherein sliding of the plunger relative to the first barrel body portion actuates the first portion of the sealing device to the radially open configuration and the second portion to the axially open configuration; and attaching a lock to the plunger to lock a position of the plunger relative to the first barrel body portion.
19. The method of any of clauses 13-18, wherein the first portion of the sealing device comprises radially compressible silicone.
20. The method of any of clauses 13-19, wherein the second portion of the sealing device comprises a polyisoprene substrate.

It should now be understood that the present disclosure relates to various mixing syringe assemblies and methods of mixing constituent materials with mixing syringe assemblies. The various embodiments provided herein may provide ready to use or easily assembled syringe assemblies for easily mixing components. Moreover, embodiments as provided herein may assist in maintaining sterility and/or constituent material integrity, while improving ease of mixing and delivery.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A mixing syringe assembly comprising:
a first barrel body portion defining a first constituent material section;
a second barrel body portion defining a second constituent material section, the first barrel body portion slidably disposed within the second barrel body portion; and
a sealing device coupled to a distal end of the first barrel body portion, the sealing device selectively providing a fluid path between the first barrel body portion and the second barrel body portion, the sealing device comprising a first portion defining a fluid passageway which is selectively actuatable between a radially closed configuration and a radially open configuration and a second portion being axially translatable between an axially closed configuration and an axially open configuration.

2. The mixing syringe assembly of claim 1, further comprising a plunger, wherein advancing the plunger within the first barrel body portion actuates the first portion of the sealing device to the radially open configuration and the second portion to the axially open configuration.

3. The mixing syringe assembly of claim 1, wherein the first portion of the sealing device comprises radially compressible silicone.

4. The mixing syringe assembly of claim 1, wherein the second portion of the sealing device comprises a polyisoprene substrate.

5. The mixing syringe assembly of claim 1, wherein the sealing device comprises a housing coupled to an end of the first barrel body portion, wherein the first portion is positioned within the housing.

6. The mixing syringe assembly of claim 5, wherein the sealing device further comprises an O-ring mounted to the housing and in sliding contact with the second barrel body portion.

7. A mixing syringe assembly comprising:
a first barrel body portion defining a first constituent material section;
a second barrel body portion defining a second constituent material section, the first barrel body portion slidably disposed within the second barrel body portion;
a sealing device coupled to a distal end of the first barrel body portion, the sealing device selectively providing a fluid path between the first barrel body portion and the second barrel body portion, the sealing device comprising a first portion defining a fluid passageway which is selectively actuatable between a radially closed configuration and a radially open configuration and a second portion being axially translatable between an axially closed configuration and an axially open configuration;
a plunger axially moveably within the first barrel body portion; and
a lock configured to brace the plunger in a locked position.

8. The mixing syringe assembly of claim 7, wherein advancing the plunger within the first barrel body portion actuates the first portion of the sealing device to the radially open configuration and the second portion to the axially open configuration.

9. The mixing syringe assembly of claim 7, wherein the first portion of the sealing device comprises radially compressible silicone.

10. The mixing syringe assembly of claim 7, wherein the second portion of the sealing device comprises a polyisoprene substrate.

11. The mixing syringe assembly of claim 7, wherein the sealing device comprises a housing coupled to an end of the first barrel body portion, wherein the first portion is positioned within the housing.

12. The mixing syringe assembly of claim 11, wherein the sealing device further comprises an O-ring mounted to the housing and in sliding contact with the second barrel body portion.

13. A method of assembling a mixing syringe assembly, the method comprising:
coupling a sealing device to a distal end of a first barrel body portion, wherein the sealing device comprises a first portion defining a fluid passageway which is selectively actuatable between a radially closed configuration and a radially open configuration and a second portion being axially translatable between an axially closed configuration and an axially open configuration; and
inserting the sealing device and the first barrel body portion into a second barrel body portion, such that the sealing device and the first barrel body portion are slidably disposed in the second barrel body portion.

14. The method of claim 13, further comprising inserting a plunger into the first barrel body portion, wherein sliding of the plunger relative to the first barrel body portion actuates the first portion of the sealing device to the radially open configuration and the second portion to the axially open configuration.

15. The method of claim 13 further comprising placing a first constituent material into the first barrel body portion.

16. The method of claim 15, further comprising placing a second constituent material into the second barrel body portion, the second constituent material being different from the first constituent material.

17. The method of claim 16, wherein the first constituent material is a liquid and the second constituent material is a powder.

18. The method of claim 13, further comprising:
inserting a plunger into the first barrel body portion, wherein sliding of the plunger relative to the first barrel body portion actuates the first portion of the sealing device to the radially open configuration and the second portion to the axially open configuration; and
attaching a lock to the plunger to lock a position of the plunger relative to the first barrel body portion.

19. The method of claim 13, wherein the first portion of the sealing device comprises radially compressible silicone.

20. The method of claim 13, wherein the second portion of the sealing device comprises a polyisoprene substrate.
